(19) United States
(12) Patent Application Publication
(10) Pub. No.: US 2005/0238600 A1
(43) Pub. Date: Oct. 27, 2005

(54) Method for using zinc oxide hair lotion

(76) Inventor: Wu Kuan Lien, Taichung City (TW)

Correspondence Address: CHARLES E. BAXLEY, ESQ., 90 JOHN STREET, THIRD FLOOR, NEW YORK, NY, 10038, US

(21) Appl. No.: 10/841,165

(22) Filed: May 6, 2004

(30) Foreign Application Priority Data
Apr. 26, 2004 (CN) 093104856

(51) Int. Cl. A61K 7/44; A61K 7/135; A61K 33/32

(52) U.S. Cl. 424/70.5; 424/641

(57) ABSTRACT

A method for using zinc oxide hair lotion includes the following steps, 1) apply zinc oxide hair lotion to user's hair 2) apply waving lotion to the user's hair 3) apply the neutralizer to the user's hair 4) style the hair. Through the above steps, the zinc oxide can ooze into the hair tissue, such that the zinc oxide not only can reduce the hydrogen nitride in the waving lotion, but also can protect the hair tissue from bacteria and ultraviolet light. Furthermore, the zinc oxide not only can absorb infrared rays to speed up the recovery rate of the hair tissue, but also can prevent electrostatic hair.

### BACKGROUND OF THE INVENTION

1. Field of the Invention

The present invention relates to a method for using zinc oxide hair lotion, and more particularly to a method for using zinc oxide hair lotion to protect hair tissue from bacteria and ultraviolet light, and also to speed up the recovery rate of the hair tissue and prevent electrostatic hair, furthermore, the zinc oxide can eliminate the smell of the waving lotion and neutralizer.

2. Description of the Prior Arts

With the fast growth of the hairstyling techniques, many kinds of chemical hair waving lotions and methods for styling hairs have been used in the hairstyling industry. The methods for using the chemical hair lotions are different, however, getting the best effect is the user's most concern. In this case, how to find a better hairstyling method has become the most concern of the professional hair designers and chemical hair lotion producers, and also it is the motive of the present invention.

Generally, the hairstyling method is using chemical lotion to change the hairstyle. Hair is made of amino acids, and hairstyle change is normally decided by the following factors:
1, disulfide bond: the S—S covalent bond between the cystine in the hair cuticle;
2, bond: the ion bond between the acid and the base groups in the amino acids;
3, hydrogen bond: produced by the amino acids.

Although, the hydrogen bond is weaker than the covalent bond and the ion bond, the quantity of the hydrogen bond is much greater than that of the covalent bond and the ion bond, and in this case, the hairstyle (shape of the hair) is fixed when the hair is dry. When the hair is wet, the hair will lose its shape since the hydrone breaks the hydrogen bond. When the hair is dry, the hydrogen bond is reproduced so that the hair regains its shape. This transient hairstyle change cannot satisfy the user's need. The conventional hair waving technique can gain a long time of hairstyle change, it breaks the disulfide bond and then restyle the hair, and finally reproduce the disulfide bond. Since the shape of the new hairstyle is maintained by the S—S covalent bond which is stronger than the hydrogen bond, it will not be changed in shape whatever the hair is wet or dray.

Waving lotion and neutralizer must be used in hair styling, the waving lotion is an alkaline solution with thioglycolic acid, which can transfer the hydrogen atom to the sulfur atom of the cystine, and brake the disulfide bond and form cysteine. At the moment, the strength for maintaining the shape of the hairstyle doesn't exist, so the hair can be arbitrarily reshaped and restyled. After that, the neutralizer (diluted hydrogen peroxide solution) is used to reproduce the disulfide bond, and thus the hairstyle is shaped.

This conventional method has been widely used for long time, however, there are still some defects need to be improved as follows:
1, since the hair waving lotion and the neutralizer will cause harm to the hair, the problems of hair losing and color fading may happen after the hair is hurt by the ultraviolet rays of the sunlight.
2, the hair waving lotion and the neutralizer weakens the activity of the hair tissue, the hair root is susceptible to bacterial infection, and the hair tissue is difficult to recover by itself.
3, the hair will be relative dry cause it is affected by the hair waving lotion and the neutralizer, electrostatic phenomenon may happen, so the hair is difficult to be combed.
4, all hair-waving lotions have strong smell and it will discomfort the hair designer and the related people.

The present invention has arisen to mitigate and/or obviate the afore-described disadvantages of the conventional methods for hair styling.

### SUMMARY OF THE INVENTION

The primary object of the present invention is to provide a method for using zinc oxide hair lotion in hair styling, which includes the steps: 1) apply zinc oxide hair lotion to user's hair 2) apply waving lotion to the user's hair 3) apply the neutralizer to the user's hair 4) style the hair, wherein the zinc oxide is used to eliminate the smell of the hair waving lotion and the neutralizer and to protect the hair tissue from bacteria and ultraviolet light, furthermore, the zinc oxide also can speed up the recovery rate of the hair tissue and prevent electrostatic hair.

The secondary object of the present invention is to provide a method for using zinc oxide hair lotion in hair styling, wherein the zinc oxide can ooze into the hair, so as to maintain the hair style for a long period of time.

The present invention will become more obvious from the following description when taken in connection with the accompanying drawings, which shows, for purpose of illustrations only, the preferred embodiment in accordance with the present invention.

### BRIEF DESCRIPTION OF THE DRAWINGS

FIG. 1 is a flow chart for illustrating the method of the present invention;

FIG. 2 is an illustrative diagram of illustrating the step of laminating the hair with zinc oxide lotion;

FIG. 3 is an illustrative diagram of illustrating the step of laminating the hair with hair waving lotion;

FIG. 4 is an illustrative diagram of illustrating the step of laminating the hair with neutralizer;

FIG. 5 is an illustrative diagram of illustrating the step of styling the hair.

### DETAILED DESCRIPTION OF THE PREFERRED EMBODIMENT

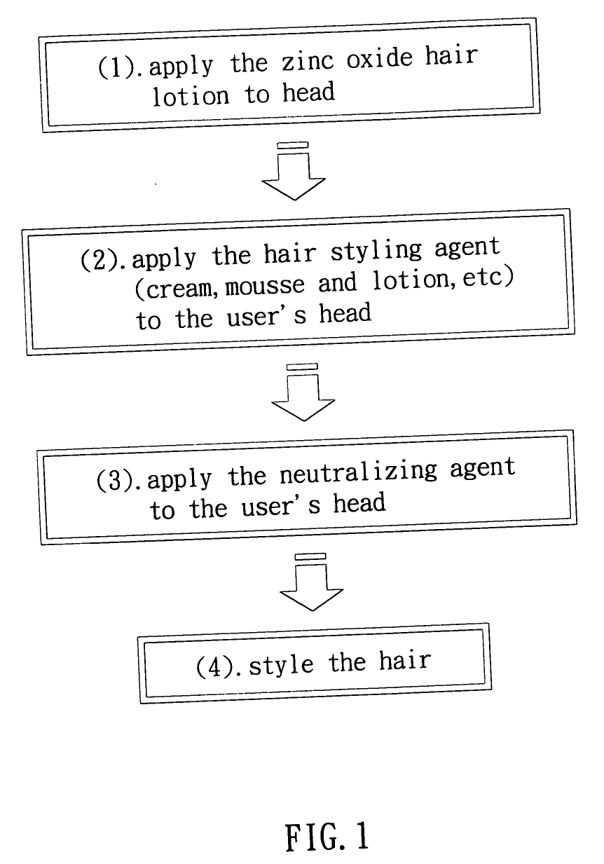

Referring to FIG. 1, which is a flow chart for illustrating a method of using zinc oxide hair lotion, which includes the following steps:
1) apply zinc oxide hair lotion to user's hair: apply the zinc oxide hair lotion containing nanophase zinc oxide particles to user's hair;
2) apply waving lotion to the user's hair: apply the waving lotion with alkaline solution to user's hair so as to open the hair cuticle, the hydrogen atom is transferred to the sulfur atom of the cystine so as to break the break the disulfide bond, and synchronously the nanophase zinc oxide particles enter into the hair cuticle, at the same time, partial ZnO is affected by the alkaline waving lotion and will produce derivatives;
3) apply neutralizer to the user's hair: apply the neutralizer to user's hair so as to close the hair cuticle and the disulfide bond is reproduced, at this moment, the nanophase zinc oxide particles are evenly and fully distributed in-and-out side the hair cuticle, with the help of the acid neutralizer, the ZnO derivatives are reduced to ZnO again;
4) style the hair: a new hair style is achieved.

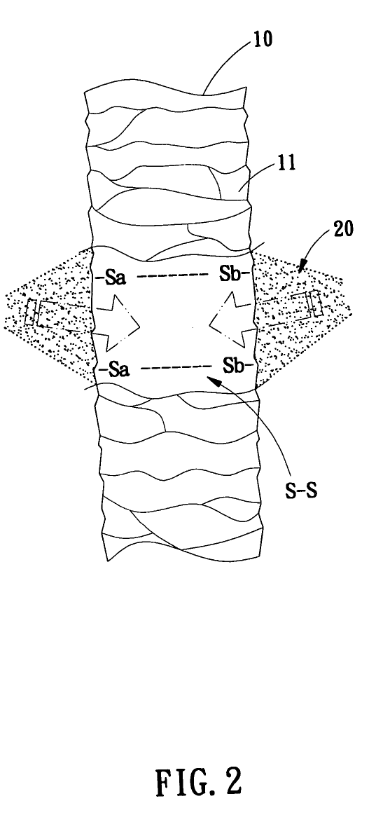

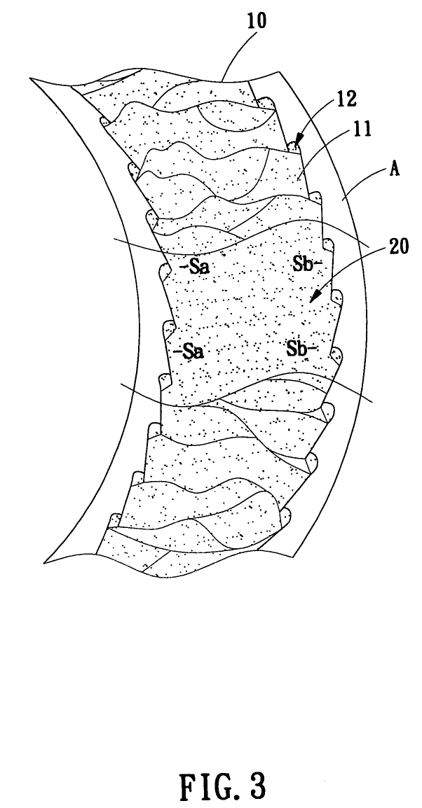

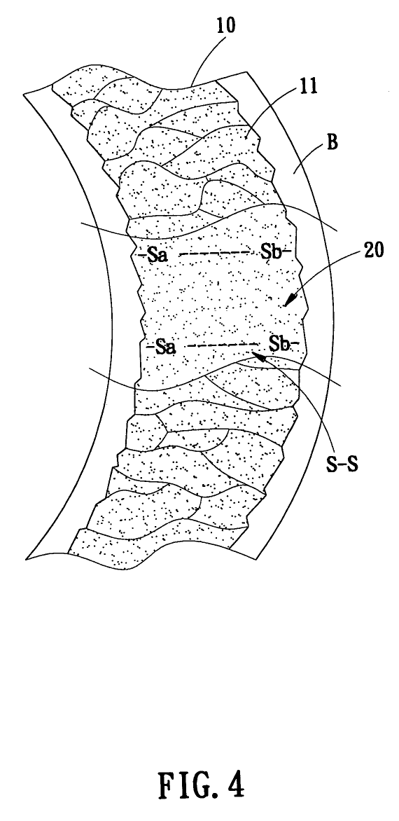

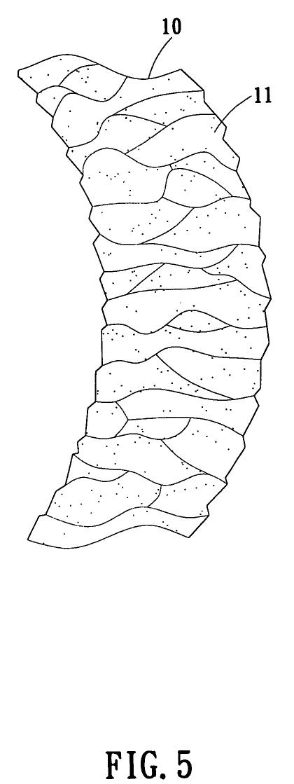

Referring to FIGS. 2-5, which are illustrative diagrams for illustrating the method of using zinc oxide hair lotion in accordance with one aspect of the present invention, the steps of the methods for using zinc oxide hair lotion are explained in details as follows:
step 1, apply zinc oxide hair lotion to user's hair: apply the ZnO hair spray containing nanophase zinc oxide particles 20 to hair surface 10, the zinc oxide particles 20 will be fully distributed throughout the surface of the hair 10 and gathered at the openings of the hair cuticle since its dimension is approximately 50 nanometers;
step 2, apply waving lotion to the user's hair: apply the waving lotion A with alkaline solution to user's hair 10 so as to open the hair cuticle 11 and forming clearances 12, at this moment, the hydrogen atom is transferred to the sulfur atom (Sa˜Sb) of the cystine so as to break the break the disulfide bond (S—S), and synchronously the nanophase zinc oxide particles 20 enter into the hair cuticle 11 via the clearances 12, at the same time, partial ZnO is affected by the alkaline waving lotion and will produce derivatives;
Step 3: apply neutralizer to the user's hair: apply the neutralizer B to user's hair so as to close the clearances 12 of the hair cuticle and the disulfide bond (S—S) is reproduced, at this moment, the nanophase zinc oxide particles 20 are synchronously enclosed the clearances 12 of the hair cuticle 11, with the help of the acid neutralizer, the ZnO derivatives are reduced to ZnO again, thus the nanophase zinc oxide particles 20 are evenly and fully distributed in-and-out side the hair 10;
Step 4: style the hair: the hair 10 is styled after the disulfide bond (S—S) is reproduced.

The nanophase zinc oxide particles 20 distributed in-and-out side the clearances 12 of the hair cuticle 11 can effectively absorb the ultraviolet rays and the infrared rays, it can protect the hair from UVA, UVB. Furthermore, infrared rays absorption can help to speed up the recovery rate of the hair tissue, and also can prevent the skin from ultraviolet radiation.

It will be noted that, in the sunlight (especially in the ultraviolet rays), in the water or in the air, the nanophase zinc oxide particles 20 can automatically produce freely moving negative electrons (e−) while leaving with empty cells with positive electricity (h+), these empty cells can activate the oxygen in the air and change it into active oxygen, the active oxygen is very chemically active and can react with many organic substances (including the bacteria), such that most of the bacteria and virus on the hire 10 will be killed. According to related quantitative tests that the bactericidal rate (in 5 minutes) will reach 99.93% on the condition that the concentration of the nanophase zinc oxide particles 20 is 1%.

In addition, the present invention embeds the nanophase zinc oxide particles 20 in the hair by opening the hair cuticle 11, such that the nanophase zinc oxide particles 20 in the hair not only can prevent electrostatic hair, but also can reduce the smell of the hydrogen nitride generated from the waving lotion A.

While we have shown and described various embodiments in accordance with the present invention, it should be clear to those skilled in the art that further embodiments may be made without departing from the scope of the present invention.

1. A method of using zinc oxide hair lotion comprising the following steps:
step 1, applying zinc oxide hair lotion to a user's hair: applying the zinc oxide hair lotion containing zinc oxide particles to the user's hair;
step 2, applying waving lotion to the user's hair: applying the waving lotion with alkaline solution to the user's hair so as to open hair cuticle, the waving lotion breaking disulfide bond, synchronously the zinc oxide particles entering into clearances of the hair cuticle;
step 3, applying neutralizer to the user's hair: applying the neutralizer to user's hair so as to close the hair cuticle, and reproducing the disulfide bond, meanwhile, the zinc oxide particles are enclosed in the clearances of the hair cuticle and the zinc oxide particles distributed in-and-out side the hair;
step 4, styling the hair: a new hair style is achieved after reproduction of the disulfide bond.

2. The method of using zinc oxide hair lotion as claimed in claim 1, wherein the zinc oxide particles in the step 1 are nanophase zinc oxide particles.
